(19) United States
(12) Patent Application Publication
(10) Pub. No.: US 2026/0049048 A1
(43) Pub. Date: Feb. 19, 2026

(54) METHOD FOR THE PREPARATION OF 4,4'-DICHLORODIPHENYL ETHER

(71) Applicant: TRE TAU ENGINEERING S.R.L., Torino (IT)

(72) Inventors: Mikhail Yurievich Kolobov, Ekaterinburg (RU); Nikolai Beliaev, Ekaterinburg (RU); Maria Kosterina, Yekaterinburg (RU); Francesco Taiariol, San Giusto Canavese (TO) (IT)

(73) Assignee: TRE TAU ENGINEERING S.R.L., Torino (IT)

(21) Appl. No.: 19/102,020

(22) PCT Filed: Aug. 2, 2023
(86) PCT No.: PCT/EP2023/071398

§ 371 Date: Feb. 7, 2025

(30) Foreign Application Priority Data
Aug. 11, 2022 (IT) 102022000017181

(51) Int. Cl. C07C 41/22; C07C 43/29

(52) U.S. Cl. CPC C07C 41/22; C07C 43/29; C07C 2601/16

(57) ABSTRACT

The invention relates to a process for the preparation of 4,4′-dichlorodiphenyl ether used as a starting substance in chemical synthesis including polymer synthesis. The method includes solvent-free treatment of the diphenyl ether with sulfuryl chloride. The reaction is carried out in a closed autoclave at 115-125° C. for 16-20 h. The isolation of the final product is done by fractional distillation of the resulting mixture of isomers. If necessary, additional crystallization is used, which gives an increased purity of the product (>95%). The method of the invention allows to increase the yield of the target product, to reduce the total time of the process and the consumption.

### FIELD OF THE INVENTION

The invention relates to a process for the preparation of 4,4′-dichlorodiphenyl ether used as a starting material in chemical synthesis, including polymer synthesis.

### BACKGROUND OF THE INVENTION

A number of methods are known for the synthesis of 4,4′-dichlorodiphenyloxide by chlorination of diphenyloxide.

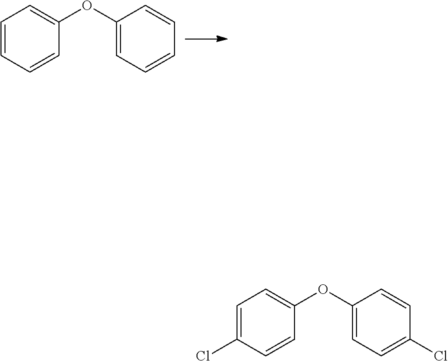 [SMILES: c1ccc(Oc2ccccc2)cc1 ; Clc1ccc(Oc2ccc(Cl)cc2)cc1]

Chlorine in acetic acid [1], sulphuryl chloride [2], chlorodimethylsulphonium chloride [3], benzene sulphonyl chloride in the presence of AlCl₃ [4] can be used as chlorinating agent. The advantage of this group of methods is the availability of the starting material and the simplicity of the methodology. The main drawback is the formation of other isomers together with para-chloro derivatives, which decreases the yield and complicates the separation of the final product. In the processes disclosed in references 3 and 4, using rather complicated reagents as chlorine sources, the selectivity of reaction and consequently the yield of the final product (in [3]—85%, [4]—90%) were increased. However, at the same time, the high cost of these reagents reduces the possibility of industrial application of these methods.

The closest prior art document is USSR AS SU 170 525 A1, dated Apr. 23, 1965, disclosing a method of producing 4,4′-dichlorodiphenyloxide by chlorination of diphenyloxide with sulfuryl chloride. According to said document, the target product is prepared according to the following scheme:

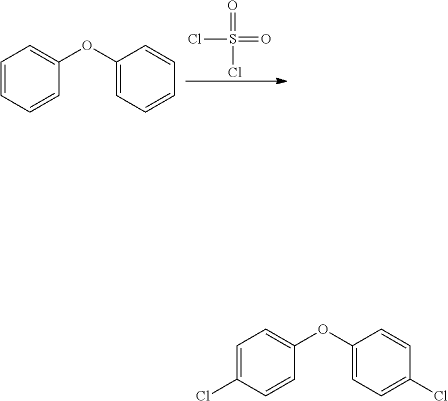

The following procedure is described in USSR AS SU 170 525 A1. In a four-neck flask, equipped with stirrer, thermometer, dropping funnel, reflux condenser, 172 g (1 mol) of pure diphenyloxide is introduced. At stirring, the reaction mass is heated on an oil bath to 80° C. and at this temperature 145 g (1.07 mol) of sulphuryl chloride is added to the flask in 30 minutes. The reaction mixture is stirred at 120° C. for 6 hours. Then the mass is cooled down to 80° C. A second portion of sulphuryl chloride (145 g, 1.07 mol) is added, and the reaction mass is stirred at 120° C. for 6 hours more. For isolation of the final product the reaction mixture is distilled in vacuum.

In an attempt to reproduce this technique in a large series of experiments, it was found that:
the temperature of the reaction mixture does not reach the required value during the reaction.
it is required twice as much sulphuryl chloride as stated in the prior document.
it takes at least 10 days to complete the reaction.
the yield of the final product does not reach the stated value, being around 35-40%.

This is explained by the fact that if sulfuryl chloride with a boiling point of 69° C. is present in the reaction mixture in an open vessel, the temperature of the mixture will decrease due to the boiling of the reagent. At a lower temperature, the reaction is much slower, and a larger excess of sulphuryl chloride is required (i.e., part of it is in the vapour phase and does not take part in the reaction). In addition, the loss of sulphuryl chloride due to entrainment through the reflux condenser increases even more the process time. Thus, it is not possible to achieve the stated reaction conditions, if the reaction is carried out in an open vessel with a reflux condenser.

### DESCRIPTION OF THE INVENTION

The present invention aims at overcoming these disadvantages and brings about the technical effect of increasing the yield of the target product while decreasing both the total time of the process and the sulphuryl chloride consumption.

In order to achieve the above-mentioned technical results, a process for the preparation of 4,4′-dichlorodiphenyloxide by chlorination of diphenyloxide with sulphuryl chloride in a closed autoclave at 110-130° C., preferably at 115-125° C. is proposed.

A general scheme of the process, including side reactions, is shown below:

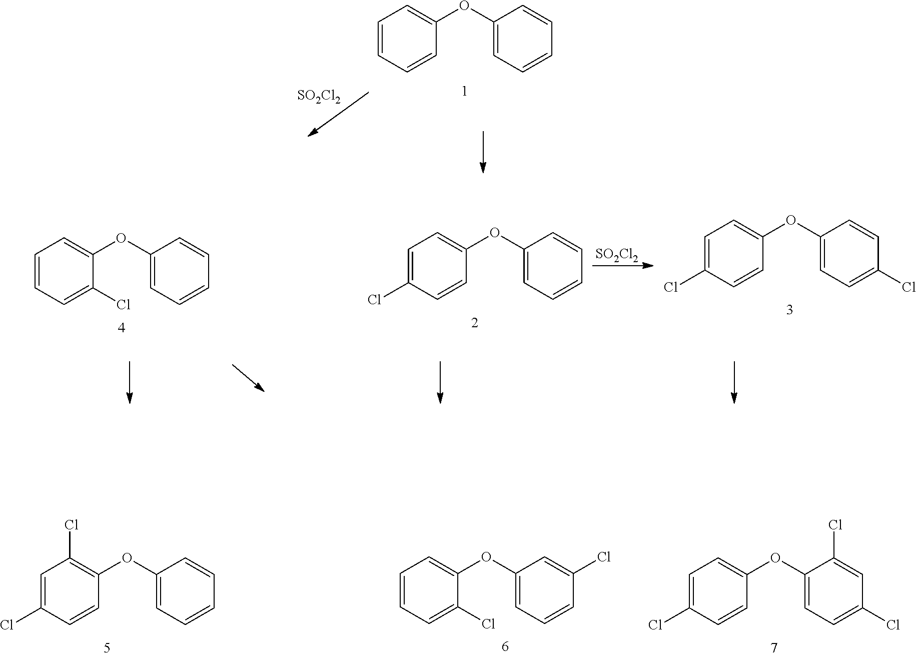

According to the proposed method, 4,4′-dichlorodiphenyloxide (3) is obtained in a closed autoclave (made of acid-resistant material or coated with it, for example, with teflon coating) at temperature 110-130° C., preferably 115-125° C. for 12-24 hours, preferably for 16-20 hours. The reaction is finished when the content of the intermediate product monochlorodiphenyloxide 2 in the reaction mixture is less than 5%.

Two variants of the process are possible. In the first variant, the whole amount of sulphuryl chloride is loaded at once and the mixture is heated for 16-20 hours. In the second variant at first ½ part of sulfuryl chloride is loaded, kept for 12-16 hours, cooled, pressure is released, the second half of sulfuryl chloride is loaded and kept for 12-16 hours more. Although taking more time, the second variant allows to reduce the pressure in the autoclave and, accordingly, to increase its loading factor.

After completion of the reaction the autoclave is cooled, the pressure is released, and the reaction mixture is distilled under vacuum. If a strongly acidic environment is undesirable for the distillation equipment, the reaction mixture is diluted with methylene chloride (e.g., about 1:1) before distillation, washed with a diluted (e.g., about 5%) alkaline solution and the solvent is evaporated. If a highly efficient distillation apparatus can be used, a product with a purity, determined by the efficiency of the distillation, is obtained. In other cases, the mixture of products containing 4,4′-dichlorodiphenyloxide at least 70% is frozen to 0-5° C. and kept at this temperature for 24 hours. The precipitate is filtered off, pressed off and 4,4′-dichlorodiphenyloxide with a purity of at least 95% is obtained.

The invention is illustrated by the following examples.

### Example 1

Diphenyloxide (25 ml, 26.8 g, 0.153 mol) and sulphuryl chloride (28 ml, 46.8 g, 0.346 mol) are loaded into 60 ml autoclave with PTFE liner, closed and heated on glycerol bath at 120° C. for 20 hours. In order to allow precise tightness control (i.e., absence of gas bubbles from under the lid), the lower edge of the autoclave lid should be below the glycerol level in the bath. The autoclave is cooled down, opened, the reaction mixture is poured into a flask, and dichloromethane (50 mL) is added, followed by a 5% NaOH solution to neutral reaction. The resulting mixture is stirred intensively for 10 minutes. After settling, the organic layer is separated, washed with water 2 times, dried over anhydrous Na₂SO₄. To improve the separation of the layers, sodium chloride can be added to the aqueous layer. Then the organic phase is evaporated under vacuum. The residue is analyzed by GC. According to GC data the mixture contains: diphenyloxide-0%, monochlorodiphenyloxide 2—1%, dichloro derivative 5—19%, 4,4′-dichlorodiphenyloxide—63.7%, trichlorodiphenyloxide 7—14%. This mixture is distilled in vacuum (e.g., 5 torr) and the fraction with boiling point 155-160° C. is collected. It is left overnight in a refrigerator at 0-5° C. The precipitate is filtered off, pressed off, and the filtrate is placed in the refrigerator for another 24 hours. An additional portion of the sediment is also filtered, pressed off and added to the first fraction. The yield of 4,4′-dichlorodiphenyloxide is 19.4 g (0.081 mol, 53%).

### Example 2

Diphenyloxide (25 ml, 26.8 g, 0.153 mol), sulphuryl chloride (14 ml, 23.4 g, 0.173 mol) are loaded into 60 ml autoclave with PTFE liner, closed and heated in glycerol bath at 120° C. for 16 hours. The lower edge of the autoclave lid should be below the glycerol level in the bath. This allows the tightness control (absence of gas bubbles from under the lid). The autoclave is cooled down, opened, another portion of sulfuryl chloride (14 ml, 23.4 g, 0.173 mol) is added, closed and heated for 16 more hours. The autoclave is cooled, opened and the final product is isolated according to example 1.

### REFERENCES

1. Brewster, Stevenson [Journal of the American Chemical Society, 1940, vol. 62, p. 3144]
2. SU170525
3. Olah, George A.; Ohannesian, Lena; Arvanaghi, Massoud [Synthesis, 1986, #10, p. 868-870].
4. Kamigata, Nobumasa; Satoh, Takeshi; Yoshida, Masato; Matsuyama, Haruo; Kameyama, Masayuki [Bulletin of the Chemical Society of Japan, 1988, vol. 61, p. 2226-2228].

1. Process for the preparation of 4,4′-dichlorodiphenyloxide, said process comprising
chlorinating diphenyloxide with sulphuryl chloride, wherein said chlorinating step is carried out in a closed autoclave at 110-130° C. for 12-24 h to obtain 4,4′-dichlorodiphenyloxide, and
separating and purifying said 4,4′-dichlorodiphenyloxide.

2. Process according to claim 1, wherein the chlorinating step of diphenyloxide with sulphuryl chloride is carried out in a closed autoclave at 115-125° C. for 16 20 h.

3. Process according to claim 1 wherein said 4,4′-dichlorodiphenyloxide is distilled and crystallised at 0-5° C., filtered and pressed.

4. Process according to claim 1 wherein a whole amount of sulphuryl chloride is loaded at once and a mixture of said whole amount of sulphuryl chloride and diphenyloxyde is heated for 16-20 hours.

5. Process according to claim 1 wherein a first ½ part of sulfuryl chloride is first loaded, kept for 12-16 hours, cooled, pressure is released, and a second half of sulfuryl chloride is loaded and kept for further 12-16 hours.
